(19) United States
(12) Patent Application Publication
(10) Pub. No.: US 2012/0037155 A1
(43) Pub. Date: Feb. 16, 2012

(54) Novel Auxiliary Chamber for Inhaled-Drugs

(76) Inventors: Chung-Chi Huang, Taipei City (TW); Jui-Ying Fu, Guishan Township (TW); Ching-Yang Wu, Linkou Township (TW); Yun-Hen Liu, Guishan Township (TW); Po-Jen Ko, Taipei City (TW)

(21) Appl. No.: 12/853,410

(22) Filed: Aug. 10, 2010

(51) Int. Cl. A61M 16/10

(52) U.S. Cl. 128/203.12

(57) ABSTRACT

An auxiliary chamber for inhaled-drugs includes a hollow tube body and a hollow side tube; and a unidirectional air valve which only allows air from an inlet flowing to an outlet end is located at the inlet end of the hollow tube body, wherein the outlet end of the hollow tube body is connected to an endotracheal tube. The hollow side tube connects with a lateral hole of the hollow tube body at one end, and a special universal adapter at the other end. The special universal adapter has a small through hole which is used to accommodate an inhaler therein to release inhaled drugs in the inhaler into the hollow tube body, and the inhaled drugs moves from the inlet end with air flow to the outlet end after entering the hollow tube body to help patients with respiratory failure take medicine.

### FIELD OF THE INVENTION

This invention relates to an auxiliary chamber for inhaled-drugs, and more particularly to provide a novel auxiliary chamber which helps patients with respiratory failure easily take inhaled-drugs.

### BACKGROUND OF THE INVENTION

Chronic Obstructive Pulmonary Disease (COPD) is a chronic respiratory disease, related to emphysema, chronic bronchitis and asthmatic bronchitis. Each of the above disease can occur individually or be accompanied by other diseases. With the progress of the disease, resistance of the airflow (in and out of the lung) increases and the airway generates some abnormal inflammatory responses to hazardous substances, causing variety of respiratory symptoms, including chronic cough, sputum, dyspnea, air exchange problems, limited respiratory capacity and recurrent respiratory tract infection.

Chronic obstructive pulmonary disease is a very common disease, the mortality rate of which is the sixth globally, the fourth in the US, and the eleventh in Taiwan. Furthermore, according to most current assessment conducted by WHO, the chronic obstructive pulmonary disease would likely rank the third in the mortality rate globally in 2030. Clinical studies indicate that long-term smoking is the primary factor causing chronic obstructive pulmonary disease. In addition, frequent exposure to respiratory irritants and dusty places, respiratory infections and genetic inheritance (lack of alpha-1-antitrypsin; ATT) are all possible dangerous factors causing chronic obstructive pulmonary disease.

Chronic obstructive pulmonary disease is an abnormal function degenerative disease and there is no effective way to cure this disease currently. The primary goal for curing the patients with such disease is to maintain substantial degree of life quality by maintaining the patients' existing respiratory functions to the best condition. However, with the progress of the disease, the patient's lung loses the function of air exchange and enters the stage of respiratory failure, namely Stage IV of this disease. At this time, the purpose of the medical treatment is to reduce the airflow resistance in and out of the lung, and maintain ventilation within residual lung tissues to further provide adequate gas exchange to the patient. Currently, the triple therapy is the most effective medical treatment for the patients with respiratory failure, which includes inhaled steroid, long acting β₂ agonist and long acting anti-cholinergic agent. Theoretically, the patient can take all clinical inhaled drugs, however, the inhaled drugs on the market are with their own specially-designed auxiliary chambers, and most of them are designed for the patients who have not yet had respiratory failure. As to the patients having chronic obstructive pulmonary disease with respiratory failure, once the patient needs a mechanical ventilator as respiratory support for a long period of time, how to appropriately provide inhaled drugs to the patient is a challenge in current clinical practice.

Therefore, there remains a need for a new and improved auxiliary chamber to overcome the abovementioned issues to effectively help the patients with respiratory failure take the inhaled-drugs.

### SUMMARY OF THE INVENTION

Conventionally, most of the inhaled-drug auxiliary chambers are designed for the patients who have not yet had experienced respiratory failure. As to the patients having chronic obstructive pulmonary disease with respiratory failure, once the patient has to use mechanical ventilator for a long period of time, how to appropriately provide inhaled drugs to the patient is a challenge in current clinical practice.

The present invention is to provide a novel auxiliary chamber for inhaled-drugs including:

a hollow tube body with a connecting unit at an inlet end which is connected with the inspiration limb of a mechanical ventilator through a snake-shaped tube; and a unidirectional air valve which only allows airflow from the mechanical ventilation to patient's lung only; and a lateral hole located between the unidirectional air valve and the outlet end, wherein the outlet end of the hollow tube body is connected to an endotracheal tube, and

a hollow side tube connecting with the lateral hole of the hollow tube body at one end, and a special universal adapter engaged at the other end, the special universal adapter having a small through hole which is used to accommodate all inhalation drugs provider devices either a metered-dose inhaler or a new soft mist inhaler, named “Respimat” therein to release inhaled drugs into the hollow tube body, and moving from the inlet end with air flow to the outlet end after entering the hollow tube body.

Comparing with conventional techniques, the inhaled-drug auxiliary chamber in the present invention can provide appropriate inhaled drugs to the patients having chronic obstructive pulmonary disease with respiratory failure. Namely, the patients can take the inhaled-drugs more easily.

The present invention together with the above and other advantages may best be understood from the following detailed description of the embodiments of the invention illustrated in the drawings below.

### BRIEF DESCRIPTION OF THE DRAWINGS

FIG. 1 illustrates a three-dimensional view of the present invention connecting with a mechanical ventilator and an endotracheal tube.

FIG. 2 illustrates a sectional view of the present invention.

FIG. 3A illustrates a three-dimensional view of a universal adaptor in the present invention.

FIG. 3B illustrates a top view of the universal adaptor in the present invention.

FIG. 4 depicts a schematic view of a use case for assembling a metered-dose inhaler in the present invention.

FIG. 5 illustrates a schematic view of a use case for assembling a soft-mist inhaler in the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

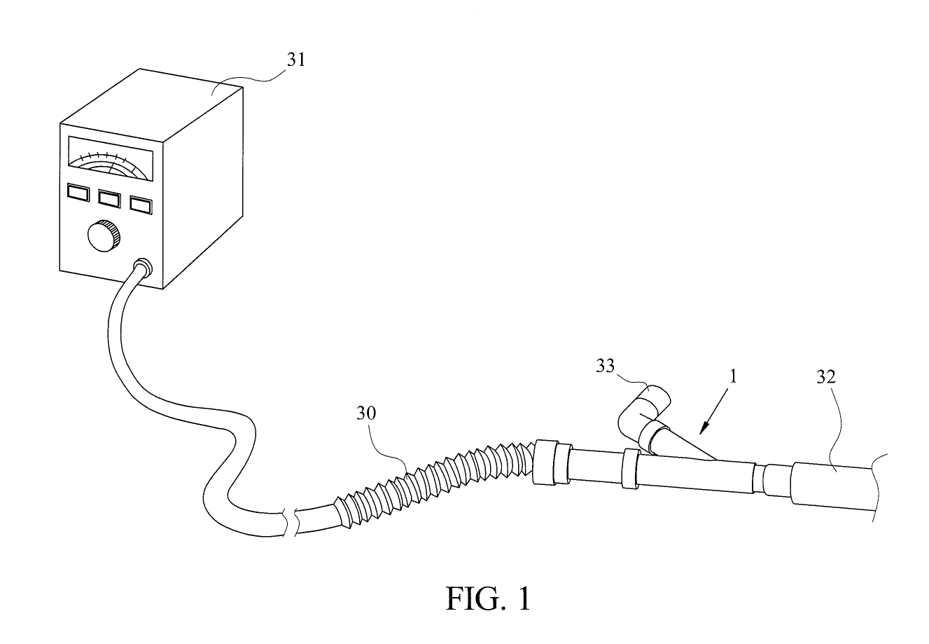

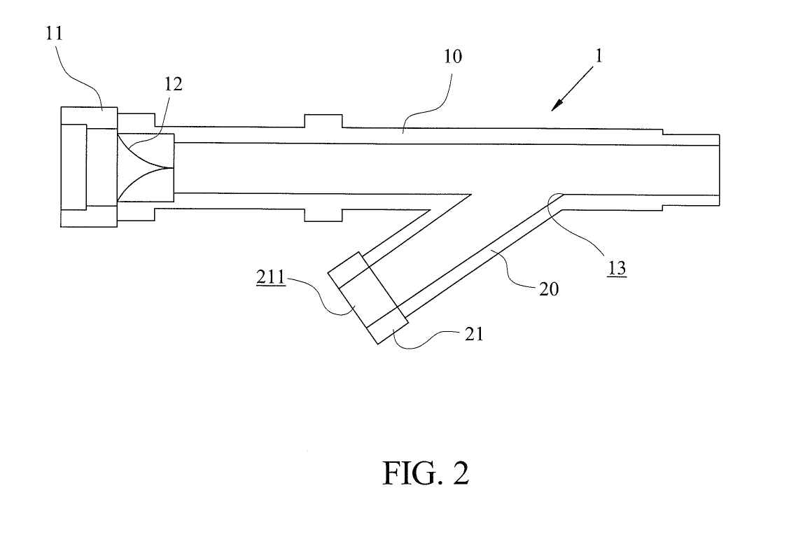

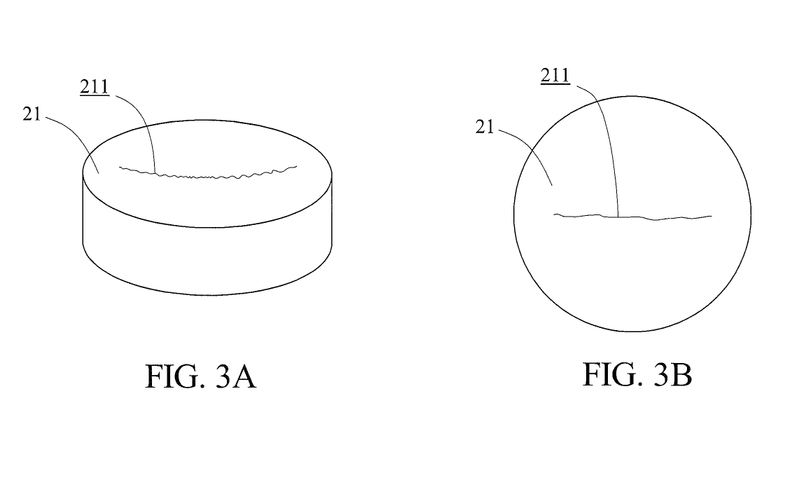

Referring to FIGS. 1 to 5, this invention is adapted to provide a novel auxiliary chamber (1) for inhaled-drugs including:

a hollow tube body (10) with a connecting unit (11) at an inlet end which is connected with a mechanical ventilator (31) through a snake-shaped tube (30); and a unidirectional air valve (12) which only allows air from the inlet end flowing to an outlet end of the hollow tube body (10); and a lateral hole (13) located between the unidirectional air valve (12) and the outlet end, wherein the outlet of the hollow tube body (10) is connected to an endotracheal tube (32), and

a hollow side tube (20) connecting with the lateral hole (13) of the hollow tube body (10) at one end, and a special universal adapter (21) at the other end, the special universal adapter (21) having a small through hole (211) which is used to accommodate an inhaler (33) therein to release inhaled drugs (40) in the inhale (33) into the hollow tube body, and the inhaled drugs (40) moving from the inlet end with air flow to the outlet end after entering the hollow tube body (10),

wherein an angle between the hollow side tube (20) and the hollow tube body (10) is 30 degrees,

wherein the special universal adapter (21) is made by silicone or rubber materials to seal one opening of the hollow side tube (20),

wherein the small through hole (211) of the universal adapter (21) is crack-shaped, and

wherein the hollow tube body (10) and the hollow side tube (20) are made by anti-static plastic material.

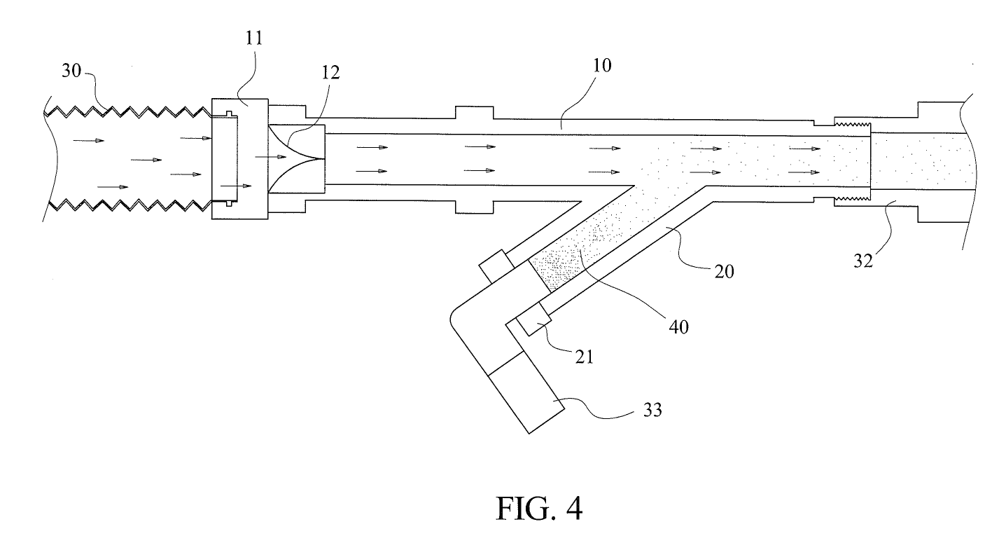

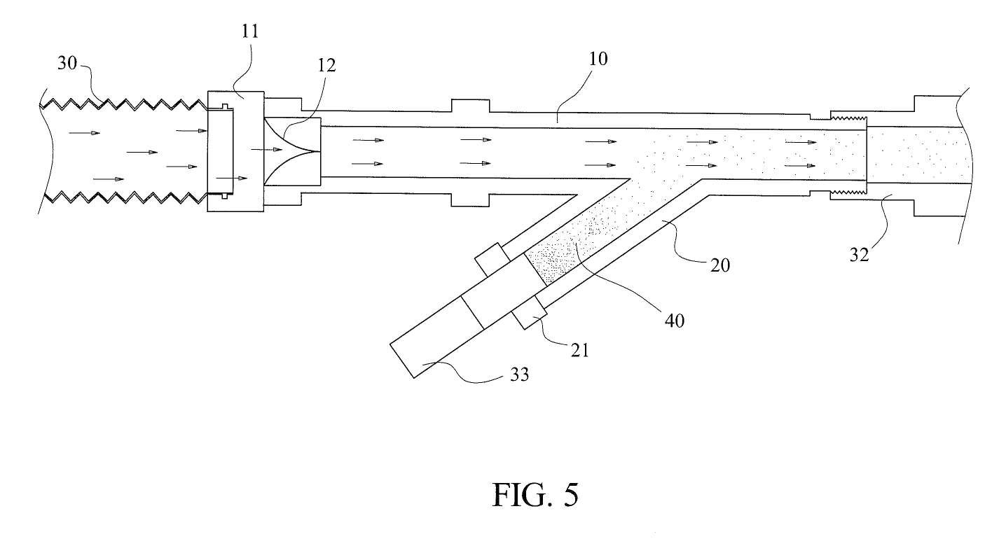

While using the auxiliary chamber in the present invention, the user can connect the inlet end of the hollow tube body (10) with the mechanical ventilator (31) via the snake-shaped tube (30), and then engage the inhaler (33) with the special universal adapter (21). Since the special universal adapter (21) is made by silicone or rubber materials, the small through holes (211) thereon are elastic, such that the inhaler (33) can be squeezed into the small through holes (211) and can be sealed therein. The user can further connect the outlet end with the endotracheal tube (32). After the assembling process, the mechanical ventilator (31) can be turned on to pump the air into the hollow tube body (10) from the inlet end, and the inhaled drugs (40) in the inhaler (33) are released into the hollow side tube (20), such that the inhaled drugs (40) can be delivered to the patient's lung along with the air pumped from the inlet end, wherein the inhaler (33) available in the market so far can be illustrated in FIG. 4 or 5, which can be easily found in the market.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. An auxiliary chamber for inhaled-drugs including:
a hollow tube body with a connecting unit at an inlet end which is connected with a mechanical ventilator through a snake-shaped tube; and a unidirectional air valve which only allows air from the inlet end flowing to an outlet end is located at the inlet end of the hollow tube body; and a lateral hole located between the unidirectional air valve and the outlet end, wherein the outlet end of the hollow tube body is connected to an endotracheal tube, and
a hollow side tube connecting with the lateral hole of the hollow tube body at one end, and a special universal adapter at the other end, the special universal adapter having a small through hole which is used to accommodate an inhaler therein to release inhaled drugs in the inhaler into the hollow tube body, and the inhaled drugs moving from the inlet end with air flow to the outlet end after entering the hollow tube body.

2. The auxiliary chamber of claim 1, wherein an angle between the hollow side tube and the hollow tube body is 30 degrees.

3. The auxiliary chamber of claim 1, wherein the special universal adapter is made by silicone materials.

4. The auxiliary chamber of claim 1, wherein the special universal adapter is made by rubber materials.

5. The auxiliary chamber of claim 1, wherein the hollow tube body and the hollow side tube are made by anti-static plastic material.
